(19) United States
(12) Patent Application Publication
(10) Pub. No.: US 2026/0176657 A1
(43) Pub. Date: Jun. 25, 2026

(54) GRANULATE COMPRISING PARTICLES OF FUNGAL BIOMASS

(71) Applicant: THE PROTEIN BREWERY B.V., Breda (NL)

(72) Inventors: Maloe KLEINE HAAR, Breda (NL); Wilhelmus Theodorus Antonius Maria DE LAAT, Breda (NL); Olumuyiwa Adetokunbo AKINTOYE, Breda (NL)

(73) Assignee: THE PROTEIN BREWERY B.V., bREDA (NL)

(21) Appl. No.: 19/109,153

(22) PCT Filed: Sep. 7, 2023
(86) PCT No.: PCT/EP2023/074670

§ 371 Date: Mar. 6, 2025

(30) Foreign Application Priority Data
Sep. 9, 2022 (EP) 22194941.5

(51) Int. Cl. C12P 7/18; A61K 9/14; C12N 1/14; C12P 7/10

(52) U.S. Cl. CPC C12P 7/18; A61K 9/14; C12N 1/14; C12P 7/10; A23V 2250/208; A61K 2300/00

(57) ABSTRACT

The invention relates to a particulate proteinaceous composition comprising granules having a protein content of at least 30 wt. % and comprising at least 80 wt. % of particles of fungal biomass, wherein the particle size distribution of the granules meets the following condition:

$${D}_{50 - grnl} \geq 50 μm ;$$

D_{x-grnl} representing the weight percentage of the granules having a diameter of less than x μm;
wherein the particle size distribution of the particles of fungal biomass comprised in the granules meets the following conditions:

$${D}_{10 - prtcl} \leq 30 μm ;$$

$$2 μm \leq {D}_{50 - gprtcl} \leq 60 μm ;$$

$${D}_{90 - prtcl} \leq 150 μm ;$$

D_{x-prtcl} representing the weight percentage of the particles of fungal biomass comprised in the granules that have a diameter of less than x μm; and
wherein the ratio D_{50-grnl}:D_{50-prtcl}≥2.0.

The functionality of proteinaceous fungal biomass is enhanced by I) strongly reducing the particle size of the fungal biomass and (ii) agglomerating the fine particles of fungal biomass so obtained into granules.

### TECHNICAL FIELD OF THE INVENTION

The present invention relates to a particulate proteinaceous composition comprising at least 80 wt. % of granules having a protein content of at least 30 wt. % and comprising at least 80 wt. % of particles of fungal biomass.

The invention also relates to a process or preparing a particulate proteinaceous composition, said process comprising:
providing fungal biomass;
subjecting the fungal biomass to size reduction treatment to produce a powder or a suspension comprising particles of fungal biomass; and
agglomerating the particles of fungal biomass.

### BACKGROUND OF THE INVENTION

Demand for edible products that can provide a high protein content which is drawn from a non-animal source is increasing. Driven by increasing awareness of personal health, edible products that include non-animal sourced components such as proteins and fibers are considered as a healthier alternative to animal protein based products. In particular, there is growing demand for edible meat substitutes that mimic meat in its composition and texture but are composed of non-animal components, which can reduce reliance animals such as cows, and reduce the carbon footprint posed by such animals.

Single-cell proteins (SCP) are an interesting alternative to meat proteins and as protein source in many other food applications such as breakfast cereals, bread, pasta, dairy, ice cream, chocolate, and soups. When fungal SCP is produced, it can be produced from sugar rich crops in a more sustainable way as compared to the production of meat protein, because SCP yields many more tonnes of protein per hectare compared to meat production and has lower nitrogen emission.

One method for producing a dietary source of protein for human food or animal feed is to produce “single cell protein” (SCP) by means of fermentation (Suman et al., 2015, Int J. Curr. Microbiol. Appl. Sci., Vol 4., No 9., pp 251-262). Fermentation in this respect is understood as the microbial conversion of carbon-rich feedstocks into protein-rich products consisting of microbial cells such as bacteria, yeasts or fungi. The use of SCP as animal feed and food ingredient brings the further advantages that microbial cells have a high content of essential amino acids. Furthermore, in particular fungal cells can be very rich in trace elements and vitamins making the fermented feedstuffs very nutritive.

SCP is already used in food products for human consumption. For instance, Quorn™ comprises a mycoprotein produced as SCP by fermentation of the fungus Fusarium venenatum. Quorn™ is available in a variety of forms such as sausages, cutlets, burgers, patties, and strips.

Fermentative production of fungal biomass typically yields a fermentation broth having a dry matter content of around 5 wt. %. This fermentation broth needs to be concentrated and optionally dried in order to render it suitable for shipping and storage under ambient conditions and to make it applicable in a broad range of food products. Concentrated or dried fungal biomass can suitably be applied in food products to provide valuable protein and dietary fibre. However, unlike some other protein sources, such as soy protein isolate, pea protein isolate and gluten, it does not impart significant texture.

US 2008/226811 describes a granulated pea protein composition having a protein content of at least 70 wt. % and a mean diameter of 150 to 300 μm.

US 2011/311599 describes a granulated powder comprising at least one vegetable protein and at least one vegetable fiber, wherein said granulated powder has:
a laser volume mean diameter D_{4,3} of between 10 μm and 500 μm, and
a dry matter content, determined after stoving at 130° C. for 2 hours, of greater than 80%.

WO 2018/029353 describes a process for producing single cell protein (SCP), the process comprising the steps of:
a) growing a thermophilic fungus in a medium containing a fermentable carbon-rich feedstock; wherein the fungus is grown in submerged culture under non-sterile conditions at a temperature higher than 45° C. and a pH of less than 3.8; and,
b) recovery of SCP from the medium in the form of biomass of the thermophilic fungus grown in step a).

US 2020/0093155 describes a method for producing a food ingredient comprising the steps of:
a. culturing filamentous fungi in growth medium;
b. harvesting filamentous fungal biomass;
c. optionally, processing the harvested filamentous fungal biomass;
d. sizing the biomass to form particles; and
e. drying the particles to form the food ingredient.

### SUMMARY OF THE INVENTION

The inventors have unexpectedly discovered that the functionality of proteinaceous fungal biomass can be enhanced by sufficiently reducing the particle size of the fungal biomass and by agglomerating the fine particles of fungal biomass so obtained into granules. These granules typically comprise at 80 wt. % of particles of fungal mass. The protein content of the granules typically is at least 30 wt. %.

Accordingly, the present invention relates to a particulate proteinaceous composition comprising at least 80 wt. % of granules having a protein content of at least 30 wt. % and comprising at least 80 wt. % of particles of fungal biomass, wherein the particle size distribution of the granules meets the following condition:

$${D}_{50 - grnl} \leq 50 μm ;$$

D_{x-grnl} representing the weight percentage of the granules having a diameter of less than x μm;
wherein the particle size distribution of the particles of fungal biomass comprised in the granules meets the following conditions:

$${D}_{10 - prtcl} \leq 30 μm ;$$

$$2 μm \leq {D}_{50 - gprtcl} \leq 60 μm ;$$

$${D}_{90 - prtcl} \leq 150 μm ;$$

D_{x-prtcl} representing the weight percentage of the particles of fungal biomass comprised in the granules that have a diameter of less than x μm; and
wherein the ratio D_{50-grnl}:D_{50-prtcl}≥2.0.

The granules according to the present invention offer the advantage that, in comparison to equally sized non-granulated particles of fungal biomass, they provide better gelling and foaming functionality, as well as a more firm texture when applied in food products, such as meat analogues.

The present invention also provides a process or preparing a particulate proteinaceous composition, said process comprising:
providing fungal biomass;
subjecting the fungal biomass to size reduction treatment to produce a powder or a suspension comprising particles of fungal biomass; wherein the particle size distribution of the particles of fungal biomass meets the following condition:

$${D}_{10 - prtcl} \leq 30 μm ;$$

$$2 μm \leq {D}_{50 - gprtcl} \leq 60 μm ;$$

$${D}_{90 - prtcl} \leq 150 μm ;$$

agglomerating the particles of fungal biomass.

### DETAILED DESCRIPTION OF THE INVENTION

A first aspect of the invention relates to a particulate proteinaceous composition comprising at least 80 wt. % of granules having a protein content of at least 30 wt. % and comprising at least 80 wt. % of particles of fungal biomass, wherein the particle size distribution of the granules meets the following condition:

$${D}_{50 - grnl} \leq 50 μm ;$$

D_{x-grnl} representing the weight percentage of the granules having a diameter of less than x μm;
wherein the particle size distribution of the particles of fungal biomass comprised in the granules meets the following conditions:

$${D}_{10 - prtcl} \leq 30 μm ;$$

$$2 μm \leq {D}_{50 - gprtcl} \leq 60 μm ;$$

$${D}_{90 - prtcl} \leq 150 μm ;$$

D_{x-prtcl} representing the weight percentage of the particles of fungal biomass comprised in the granules that have a diameter of less than x μm; and wherein the ratio D_{50-grnl}:D_{50-prtcl}≥2.0.

The term “fungal biomass” as used herein refers to organic matter that originates from fungi.

The term “protein” as used herein refers to molecules comprising a chain of at least 20 amino acids.

The term “granule” as used herein refers to particle that is an agglomerate of smaller particles, notably of smaller particles of fungal biomass. Granules (or granulates) may be produced by agglomeration techniques such as, for instance, fluid bed agglomeration, compaction, extrusion and spray drying agglomeration.

The term “fat” as used herein refers to lipids containing one or more fatty acid residues. The terms “fat” and “oil” as used herein, unless indicated otherwise, should be considered synonyms.

The term “dietary fibre” as used herein refers to polysaccharides that are not digested by human digestive enzymes. Chitin, chitosan and β-glucans are examples of dietary fibres that are found in fungi.

The term “polysaccharide” as used herein refers to a polymer comprising a long, optionally branched chain of monosaccharides, the total number of monosaccharides in the polysaccharide being at least 10.

The term “a” or “an” as used herein is defined as “at least one” unless specified otherwise.

The term “or” as used herein is defined as “and/or” unless specified otherwise.

Unless indicated otherwise, all percentages mentioned herein should be construed as percentages by weight.

The particle size distributions as referred to herein, unless indicated otherwise, are determined by laser diffraction analysis, using a Mastersizer 3000 (ex Malvern Panalytical). This technique is based on the passing of a laser beam through the dispersed particles. The angular variation in intensity of the scattered light gives information on the particle size. Larger particles scatter light at smaller angles and smaller particles scatter light at larger angles. The scattering intensity data can be used to calculate the particle size distribution.

The particle size distributions of the granules in the particulate proteinaceous composition of the present invention can be determined using a Mastersizer 3000 equipped with an Aero S dry powder dispenser. Also the particle size distribution of the particles of fungal biomass comprised in the granules can suitably be determined using this set-up. The Aero S disperses dry samples by accelerating particles through a venturi using compressed air. The particles are then pulled through the Mastersizer 3000's measurement cell using a vacuum source. The air pressure drop across the venturi is manipulated to achieve complete sample dispersion and can be controlled to within +/−0.1 bar. The particle size distribution of the granules can suitably be determined by the Mastersizer 3000 by employing dispersion pressure of 1 bar in the Aero S dispenser. The particle size distribution of the particles of fungal biomass comprised in the granules can suitably be determined by the Mastersizer 3000 by employing dispersion pressure of 4 bar in the Aero S dispenser. The use the maximum dispersion pressure of 4 bar ensure that the granules are de-agglomerated before the measurement.

The particulate proteinaceous composition of the present invention preferably does not contain any meat or meat derived components. Preferably, the composition is a vegetarian composition, more preferably a vegan composition.

The granules having a protein content of at least 30 wt. % and comprising at least 80 wt. % of particles of fungal biomass preferably constitute at least 90 wt. %, more preferably at least 95 wt. % of the particulate proteinaceous composition of the present invention. Besides these granules, the particulate proteinaceous composition may contain other components such as desiccants, glidants, micronutrients, flavouring and colouring.

According to a particularly preferred embodiment, the particulate proteinaceous composition consists of the granules comprising particles of fungal biomass.

The protein content of the granules preferably is at least 35 wt. %, more preferably 40 to 70 wt. % and most preferably 42 to 60 wt. %.

The particles of biomass in the granules preferably have protein content of at least 35 wt. %, more preferably of 40 to 70 wt. % and most preferably of 42 to 60 wt. %.

Dietary fibre is preferably contained in the particles of fungal biomass in a concentration, calculated by weight of dry matter, of 35-42 wt. %, more preferably of 30-40 wt. %.

Preferably, at least 35 wt. %, more preferably 40-75 wt. % of the dietary fibre in the particles of fungal biomass is aminopolysaccharide selected from chitin, chitosan and combinations.

Chitin preferably constitutes 15-50 wt. %, more preferably 20-40 wt. %, most preferably 25-35 wt. % of the dietary fibre in the particles of fungal biomass.

Chitosan preferably constitutes 15-50 wt. %, more preferably 20-40 wt. %, most preferably 24-34 wt. % of the dietary fibre in the particles of fungal biomass.

The dietary fibre in the particles of fungal biomass preferably contains 10-70 wt. %, more preferably 20-50 wt. % of polyglucuronic acid.

The combination of the aminopolysaccharide and polyglucuronic acid preferably constitutes at least 50 wt. %, more preferably at least 70 wt. % of the dietary fibre in the particles of fungal biomass.

The combination of fungal protein and the aminopolysaccharide preferably constitutes at least 40 wt. %, more preferably 50-70 wt. % of the dry matter that is contained in the particles of fungal biomass.

The particles of fungal biomass preferably contain, calculated by weight of dry matter, 0-15 wt. %, more preferably 0-10 wt. % and most preferably 0.5-5 wt. % of digestible carbohydrates.

The fat content of the particles of fungal biomass, calculated by weight of dry matter, preferably is in the range of 6-20 wt. %, more preferably of 6.5-12 wt. %.

The combination of protein, fat and dietary fibre preferably constitutes at least 80 wt. %, more preferably 88-99.8 wt. % of the dry matter that is contained in the particles of fungal biomass.

The particulate proteinaceous composition according to the present invention preferably has a water content of 1-15 wt. %, more preferably of 2-10 wt. % and most preferably of 3-9 wt. %.

The granulate of the present invention may suitably be prepared by fluid bed agglomeration, compaction or extrusion. Fluid bed agglomeration typically yields relatively fine granulates, whereas compaction and extrusion are mostly used to produce rather coarse granulates.

Preferably the particle size distribution of the granules is such that D_{50-grnl} is in the range of 60 to 1,500 μm, more preferably in the range of 70 to 1,200 μm and most preferably in the range of 80 to 1,000 μm.

In a preferred embodiment, the particle size distribution of the granules is such that D_{10-grnl} is at least 10 μm, more preferably in the range of 15 to 1,000 μm and most preferably in the range of 20 to 800 μm.

Advantageously, the particle size distribution of the granules is such that D_{90-grnl} is at least 100 μm, more preferably in the range of 150 to 3,000 μm and most preferably in the range of 200 to 2,000 μm.

In a preferred embodiment, the present granulate is a fine granulate having aparticle size distribution of the granules that is such that D_{50-grnl} is in the range of 60 to 500 μm, more preferably in the range of 70 to 300 μm and most preferably in the range of 80 to 250 μm.

In a preferred embodiment, the particle size distribution of the granules of the fine granulate is such that D_{90-grnl} is at least 10 μm, more preferably in the range of 15 to 100 μm and most preferably in the range of 20 to 80 μm.

Advantageously, the particle size distribution of the granules of the fine granulate is such that D_{90-grnl} is at least 100 μm, more preferably in the range of 150 to 1,000 μm and most preferably in the range of 200 to 800 μm.

According to a another preferred embodiment, the granules in the particulate composition have a span of less than 8, wherein the span equals (D₉₀-D₁₀)/D₅₀. More preferably the span of the granules is less than 5, more preferably the span is in the range of 0.8 to 4 and most preferably the span is in the range of 1.5 to 3.

The particles of fungal biomass in the granules of the present invention preferably have a particle size distribution such that D_{10-prtcl} is not more than 20 μm, more preferably in the range of 0.5 to 15 μm and most preferably in the range of 1 to 12 μm.

Preferably the particle size distribution of the particles of fungal biomass in the granules is such that D_{50-prtcl} is in the range of 2 to 40 μm, more preferably in the range of 3 to 30 μm and most preferably in the range of 4 to 25 μm.

In a preferred embodiment, the particle size distribution of the particles of fungal biomass is such that D_{90-prtcl} is in the range of 10 to 130 μm, more preferably in the range of 20 to 110 μm and most preferably in the range of 25 to 100 μm.

According to a particularly preferred embodiment, the granules of the present invention are relatively large agglomerates of the particles of fungal biomass as reflected by a ratio D_{50-grnl}:D_{50-prtcl}≥2.0. Even more preferably, said ratio is at least 3.0. Most preferably, the ratio is in the range of 4.0 to 15.0.

The fungal biomass that is contained in the particles of fungal biomass preferably is biomass of one or more fungi belonging to the class Zoopagomyceta, Mucoromyceta or Symbiomyceta. Even more preferably, the fungal biomass is biomass of one or more fungi belonging to the phylum Zoopagomycota or Mucoromycota.

In a preferred embodiment, the fungal biomass in the particles of fungal biomass is biomass of a strain of a fungal genus selected from the group consisting of Rasamsonia, Talaromyces, Penicillium, Acremonium, Humicola, Paecilomyces, Chaetomium, Rhizomucor, Rhizopus, Thermomyces, Myceliophthora, Thermoascus, Thielavia, Mucor, Stibella, Melanocarpus, Malbranchea, Dactylomyces, Canariomyces, Scytalidium, Myriococcum, Corynascus, and Coonemeria, More preferably, the fungal biomass is biomass of one or more fungi belonging to a species selected from the group consisting of Rasamsonia composticola Rasamsonia emersonii, Talaromyces emersonii, Rhizomucor miehei, Rhizomucor pusillus, Thermomucor indica-seudaticae, Thielava terricola, Thielava terrestris, Thermoascus thermophilus and a Rhizopus sp., of which the strains Rasamsonia composticola strain CBS 141695, Rasamsonia emersonii CBS 143030, Thermomucor indicae-seudaticae CBS 143027 and CBS 104.75, Rhizomucor miehei CBS 143029, Rhizomucor pusillus CBS 143028, Thermoascus thermophilus CBS 528.71, Thielavia terrestris CBS 546.86, Talaromyces emersonii CBS 393.64 and Thermothelomyces thermophila CBS 117.65 and Rhizopus sp. CBS 143160 are more preferred, of which strains CBS 141695, CBS 143030, CBS 143027, CBS 143029, CBS 143160 and CBS 143028 are most preferred.

Another aspect of the invention relates to a process or preparing a particulate proteinaceous composition, preferably a particulate proteinaceous composition according to the present invention, said process comprising:
providing fungal biomass;
subjecting the fungal biomass to size reduction treatment to produce a powder or a suspension comprising particles of fungal biomass; wherein the particle size distribution of the particles of fungal biomass meets the following condition:

$${D}_{10 - prtcl} \leq 30 μm ;$$

$$2 μm \leq {D}_{50 - gprtcl} \leq 60 μm ;$$

$${D}_{90 - prtcl} \leq 150 μm ;$$

agglomerating the particles of fungal biomass.

The fungal biomass that is subjected to a size reduction treatment in the present process may be provided in the form of e.g. flocks or a coarse powder. Preferably, the fungal biomass is provided in the form of particulate composition wherein at least 80 wt. % of the particles have a mesh-size of at least 100 μm, more preferably of a mesh-size of at least 160 μm and most preferably a mesh-size of at least 200 μm. The weight percentage of particles having a mesh-size of at least x μm is determined by employing a mesh with opening of x μm.

The fungal biomass that is subjected to size reduction treatment preferably comprises at least 10 wt. %, more preferably at least 30 wt. % and most preferably at least 50 wt. % of intact fungal cells, said weight percentage being calculated by weight of the total amount of fungal biomass.

The fungal biomass preferably has a protein content, calculated by weight of dry matter, of at least 30 wt. %, more preferably of at least 35 wt. %, even more preferably of 40 to 70 wt. % and most preferably of 42 to 60 wt. %.

The fungal biomass that is provided in the present process preferably is biomass of one or more fungi belonging to the class Zoopagomyceta, Mucoromyceta or Symbiomyceta. Even more preferably, the fungal biomass is biomass of one or more fungi belonging to the phylum Zoopagomycota or Mucoromycota.

In a preferred embodiment, the fungal biomass i is biomass of a strain of a fungal genus selected from the group consisting of Rasamsonia, Talaromyces, Penicillium, Acremonium, Humicola, Paecilomyces, Chaetomium, Rhizomucor, Rhizopus, Thermomyces, Myceliophthora, Thermoascus, Thielavia, Mucor, Stibella, Melanocarpus, Malbranchea, Dactylomyces, Canariomyces, Scytalidium, Myriococcum, Corynascus, and Coonemeria, More preferably, the fungal biomass is biomass of one or more fungi belonging to a species selected from the group consisting of Rasamsonia composticola Rasamsonia emersonii, Talaromyces emersonii, Rhizomucor miehei, Rhizomucor pusillus, Thermomucor indica-seudaticae, Thielava terricola, Thielava terrestris, Thermoascus thermophilus and a Rhizopus sp., of which the strains Rasamsonia composticola strain CBS 141695, Rasamsonia emersonii CBS 143030, Thermomucor indicae-seudaticae CBS 143027 and CBS 104.75, Rhizomucor miehei CBS 143029, Rhizomucor pusillus CBS 143028, Thermoascus thermophilus CBS 528.71, Thielavia terrestris CBS 546.86, Talaromyces emersonii CBS 393.64 and Thermothelomyces thermophila CBS 117.65 and Rhizopus sp. CBS 143160 are more preferred, of which strains CBS 141695, CBS 143030, CBS 143027, CBS 143029, CBS 143160 and CBS 143028 are most preferred.

The size reduction treatment that is used to produce a powder or a suspension comprising particles of fungal biomass preferably comprises milling or grinding.

In an advantageous embodiment of the present process, the size reduction treatment produces a powder comprising the particles of fungal biomass. Such a powder may suitably be agglomerated by means of fluid bed agglomeration, compaction or extrusion.

In another embodiment, the fungal biomass is provided in the form of wet biomass having a water content of 75 to 99 wt. % and the size reduction treatment produces a suspension of the particles of fungal biomass. The particles of fungal biomass in the suspension may suitably be agglomerated by means of, for instance, spray drying agglomeration.

According to a particularly preferred embodiment, the present process yields a particulate proteinaceous composition as defined herein before.

A further aspect of the invention relates to a food product comprising, calculated by weight of dry matter, at least 1 wt. %, preferably 10-95 wt. % of the particulate proteinaceous composition of the present invention.

Examples of food products in which the particulate proteinaceous composition of the present invention may suitably be applied include textured protein (e.g. TVP), meat analogues, meat products, soups, sauces, cereal-based food products and pet food.

Yet another aspect of the invention relates to a method of preparing the aforementioned food product, said method comprising combining the particulate proteinaceous composition of the present invention with one or more other edible ingredients.

The present invention is further illustrated by the following non-limiting examples.

### EXAMPLES

### Example 1: Production of Rhizomucor pusillus Biomass in Cake or Powder Form

For preculture Rhizomucor pusillus strain CBS 143028 was inoculated in 200 ml of a defined mineral medium at pH 5.5 containing KCl 0.17 g/L, KH₂PO₄ 1.3 g/L, Na₂HPO₄ 0.4 g/L, citric acid 0.5 gr/L, MgSO₄·7 aq 0.7 gr/L, FeSO₄·7 aq 0.03 gr/L, CaCl₂·2 aq 0.035 gr/L, ZnSO₄·7 aq 0.04 gr/L, MnCl₂·4 aq 0.004, CuSO₄·5 aq 0.0005 gr/L, CoCl₂·6 aq 0.0005 gr/L, Na2B₄O₇·10 aq 0.003 gr/L, KI 0.0003 gr/L, Na₂MoO₄·2 aq 0.0005 gr/L, 11 g Dextrose per L; 4 g (NH₄)₂SO₄ per L; and 7,5 g tartaric acid per L. The preculture was incubated for 24 hours at 46° C., in a 1 L Erlenmeyer flask with air permeable stop with baffles, in an orbital shaker at 200 rpm. The preculture was then used to inoculate a fermenter containing the defined mineral medium as described above at a pH of 3.5 and comprising 77 g Dextrose per L as C-source; 1.4 g (NH₄)₂SO₄ per L as N-source and supplemented with NH₃ as titrant. The fungus was grown in the fermenter in fed batch mode at a doubling time of 12 hours. Olive oil was continuously being fed to maintain a concentration of 50 ppm.

Fermentation broths, having reached a dry matter content ranging from 2 to 5 weight percent, were concentrated using a vibrating sieve to achieve a minimum of 10% (w/w) dry matter. The biomass is then mixed with anti-oxidant and pasteurized.

Next the sieved biomass was compressed with a hydraulic press to obtain the Rhizomucor pusillus biomass as a cake with a dry matter content of about 29% (w/w). Part of the biomass cake was further freeze dried and milled (6,000 rpm, 0.5 mm mesh size) to obtain the Rhizomucor pusillus biomass in powder form with a dry matter content of about 96% (w/w).

The compositions of the Rhizomucor pusillus biomass in cake form and powder form were analysed. The results are shown in Tables 1-3.

TABLE 1

| Composition | Biomass in cake form | Biomass in powder form |
|---|---|---|
| Moisture | 71.1 g/100 g | 4 g/100 g |
| Ash | 1 g/100 g | 3.2 g/100 g |

TABLE 2

| | Rhizomucor pusillus | Rhizomucor pusillus |
|---|---|---|
| | biomass cake | biomass powder |
| Nutritional value | (29% dm) | (96% dm) |
| Crude protein content* | 14.3 g/100 g | 47.6 g/100 g |
| Total fat content | 2.4 g/100 g | 8.1 g/100 g |
| Total dietary fiber** | 10.6 g/100 g | 34.8 g/100 g |
| Total digestible | 0.8 g/100 g | 2.4 g/100 g |
| carbohydrates | | |
| Total sugars | <0.5 g/100 g | <0.5 g/100 g |
| Energy (kJoules) | 431 kJ/100 g | 1429 kJ/100 g |
| Energy (kcal) | 103 kcal/100 g | 341 kcal/100 g |

*Based on Kjeldahl method (N*6.25).
**Determination of the content of the total number dietary fiber method; enzymatic pre-treatment, gravimetr

TABLE 3

| | Rhizomucor pusillus |
|---|---|
| | biomass cake |
| Amino acid profile | (29% dm) |
| Aspartic acid (+Asparagine) | 1.04 g/100 g |
| Threonine* | 0.52 g/100 g |
| Serine | 0.52 g/100 g |
| Glutamic acid (+Glutamine ) | 1.30 g/100 g |
| Proline | 0.39 g/100 g |
| Glycine | 0.48 g/100 g |
| Alanine | 0.63 g/100 g |
| Citrulline | >1 g/100 g |
| Valine* | 0.61 g/100 g |
| Isoleucine* | 0.52 g/100 g |
| Leucine* | 0.84 g/100 g |
| Tyrosine | 0.42 g/100 g |
| Phenylalanine* | 0.47 g/100 g |
| Lysine* | 0.80 g/100 g |
| Histidine* | 0.27 g/100 g |
| Arginine | 0.63 g/100 g |
| Cysteine | 0.12 g/100 g |
| Methionine* | 0.22 g/100 g |
| Tryptophan* | 0.15 g/100 g |

*Essential amino acid for humans.

It must be taken into account that the protein content of the Rhizomucor pusillus biomass as shown in Table 2 is based on the Kjeldahl method, which is a standard method use to analyze the protein content of different food products. The Kjeldahl method is based on the total nitrogen content and uses a standard conversion factor of 6.25 to estimate the protein content in the analyzed food product. However, a conversion factor of 6.25 may not be appropriate for certain food products.

In the Rhizomucor pusillus biomass, the conversion factor may be overestimated due to the presence of other nitrogen sources such as RNA, chitin and chitosan. The real protein content of the Rhizomucor pusillus biomass can for this reason better be estimated through amino acid analysis.

### Example 2: Dietary Fibre Composition of the Rhizomucor pusillus Biomass

The dietary fibre content and composition of the Rhizomucor pusillus biomass was determined. The results are shown in Tables 4 and 5.

TABLE 4

| Dietary Fiber (DF) | DF | Moisture | % dry | DF |
|---|---|---|---|---|
| content | g/100 g | g/100 g | matter | % dm |
| Rhizomucor pusillus | 10.6 | 71.1 | 29 | 37 |
| biomass (cake) | | | | |

TABLE 5

| | Batch 1 | Batch 2 | Batch 3 | Batch 4 | Average | Unit |
|---|---|---|---|---|---|---|
| Chitin | 8.6 | 10.8 | 9.8 | 9.9 | 9.8 | g/100 g |
| Chitosan | 9.2 | 9.5 | 8.9 | 9 | 9.2 | g/100 g |

The dry matter-based dietary fiber content of the Rhizomucor pusillus biomass is extremely high. Interestingly, the Rhizomucor pusillus biomass does not only contain chitin as fiber but contains an almost equal amount of the fiber chitosan. Chitosan is the de-acetylated form of chitin and health benefits have been described for chitosan in animals.

### Example 3: Preparation of a Fine Powder of Rhizomucor pusillus Biomass

Milled biomass powders were produced in the same way as described in Example 1 except that next to normally milled biomass powder (Powder 1) also a very finely milled biomass powder (Powder 2) was produced (18,000 rpm, 0.12 mm mesh size). The particle size distributions of the milled powders were determined by means of laser diffraction, using a Mastersizer 3000 equipped with an Aero S dry powder dispenser. The results are summarized in Table 6.

TABLE 6

| | Powder 1 | Powder 2 |
|---|---|---|
| D₁₀ (in μm) | 6.6 | 2.1 |
| D₅₀ (in μm) | 70 | 11 |
| D₉₀ (in μm) | 240 | 80 |

### Example 4: Rheological Test

The milled powders of Example 3 were combined in a ratio of 1:5 (w/w) with 25 mM Tris-HCl buffer pH 8 and stirred for 2.5 hours. Next, the suspensions were centrifuged at 5000 g and separated into a pellet and a supernatant fraction. The supernatants so obtained were each concentrated five times. The concentrated supernatants to obtained had a protein content of about 2.5 mg/mL.

The concentrated supernatants were heated to 80° C. for 20 minutes and then cooled down to 25° C. Next the breaking stress and strain of the heated samples was measured with an amplitude sweep measurement using a MCR301 rheometer from Anton Paar.

No breaking stress could be detected in the heated sample that had been produced from the supernatant of the normally milled biomass (Powder 1). The heated sample of the supernatant of the finely milled biomass powder (Powder 2), however, had a breaking stress of 55 Pa, a breaking strain of 1.18% and a storage modulus of 8000 Pa.

### Example 5: Application Test

The milled biomass powders of Example 3 were applied in vegan burgers. The compositions of the burgers are shown in Table 7.

TABLE 7

Wt. %

| | 1 | 2 | 3 | 4 |
|---|---|---|---|---|
| Powder 1 (hydrated 1:3) | 52 | 54 | | |
| Powder 2 (hydrated 1:3) | | | 52 | 54 |
| Textured wheat protein ¹ (hydrated 1:3) | 44 | 44 | 44 | 44 |
| Egg white | 4 | 2 | 4 | 2 |

¹ Lory ® Tex, ex Loryma GmbH, Germany

The burgers were prepared as follows:
The milled biomass powder and TVP were each hydrated in a volume ratio of 1:3 with tap water
The hydrated TVP and hydrated biomass powder were mixed together and the egg white was added
The mixture was mixed for 1 minute with a hand mixer at the lowest setting (1)
100 g burgers were formed
The burgers were left to settle for 10 minutes
The burgers were steamed for 20 minutes at 100° C.
The burgers were frozen for storage
The burgers were defrozen on the day of the tasting
The burgers were baked for 5 minutes at medium/high temperature

The burgers were prepared for consumption and evaluated by an expert panel. The results of the evaluation as summarized in Table 8.

TABLE 8

| | Evaluation results |
|---|---|
| Burger 1 | Acceptable texture |
| Burger 2 | Less bite, less firm, more crumbly than Burger 1 |
| Burger 3 | Nice texture. More firm and more dense than Burger 1 |
| Burger 4 | Firmer compared to Burger 2. More moisture than Burger 3 |

### Example 6: Preparation of a Granulate of Rhizomucor pusillus Biomass

Powder 2 of Example 3 is granulated by means of fluid bed agglomeration.

The granulate so obtained has a particle size distribution that meets the specification that is shown in Table 9.

TABLE 9

| | |
|---|---|
| D₁₀ (in μm) | 10-50 |
| D₅₀ (in μm) | 70-120 |
| D₉₀ (in μm) | 80-500 |

### Example 7: Application Test

Powder 2 of Example 3 and the biomass granulate of Example 6 are applied in vegan burgers having the compositions that are shown in Table 10.

TABLE 10

Wt. %

| | 1 | 2 |
|---|---|---|
| Powder 2 (hydrated 1:3) | 53 | |
| Biomass granulate (hydrated 1:3) | | 53 |
| Textured wheat protein ¹ (hydrated 1:3) | 44 | 44 |
| Egg white | 3 | 3 |

¹ Lory ® Tex, ex Loryma GmbH, Germany

The burgers are prepared for consumption and evaluated by an expert panel. The burgers are found to be very similar and to have a very nice firm texture.

### Example 8: Preparation of a Granulate of Rhizomucor pusillus Biomass

Powder 2 of Example 3 was granulated in a Process 11 twin-screw extruder (Thermo Fischer Scientific, Karlsruhe, Germany). The extrusion conditions employed are summarized in Table 11.

TABLE 11

| | | |
|---|---|---|
| During extrusion | Moisture content (%) | 47 |
| | Extrusion temperature (° C.) | 79 |
| | Cutter speed at end of extruder die (rpm) | 90 |
| After extrusion | Drying temperature (° C.) | 70 |
| | Drying time (min) | 60 |

The particle size distribution of the granulate was determined by means of laser diffraction, using a Mastersizer 3000 equipped with an Aero S dry powder dispenser, The so obtained result is shown in Table 12.

TABLE 12

| | |
|---|---|
| D₁₀ (in μm) | 513 |
| D₅₀ (in μm) | 899 |
| D₉₀ (in μm) | 1460 |
| Span (D₉₀ − D₁₀)/D₅₀ | 1.06 |

1.-15. (canceled)

16. A particulate proteinaceous composition comprising at least 80 wt. % of granules having a protein content of at least 30 wt. % and comprising at least 80 wt. % of particles of fungal biomass, wherein the particle size distribution of the granules meets the following condition:
$$D 50 - grnl \geq 50 μm ;$$
Dx-grnl representing the weight percentage of the granules having a diameter of less than x μm;
wherein the particle size distribution of the particles of fungal biomass comprised in the granules meets the following conditions:
$$D 10 - prtcl \leq 30 μm ;$$
$$2 μm \leq D 50 - gprtcl \leq 60 μm ;$$
$$D 90 - prtcl \leq 150 μm ;$$
Dx-prtcl representing the weight percentage of the particles of fungal biomass comprised in the granules that have a diameter of less than x μm; and
wherein the ratio D50-grnl:D50-prtcl≥2.0;
wherein the particle size distributions are determined using a Mastersizer 3000 equipped with an Aero S dry powder dispenser; and wherein the particle size distribution of the granules is determined by employing a dispersion pressure of 1 bar and wherein the particle size distribution of the particles of fungal biomass comprised in the granules is determined by employing a dispersion pressure of 4 bar.

17. Particulate proteinaceous composition according to claim 16, wherein the granules meet the following condition:
$$D 10 - grnl \geq 50 μm$$

18. Particulate proteinaceous composition according to claim 16, wherein the granules meet the following condition:
$$D 90 - grnl \geq 100 μm$$

19. Particulate proteinaceous composition according to claim 16, wherein composition has a water content of 1-15 wt. %.

20. Particulate proteinaceous composition according to claim 16, wherein the fungal biomass is biomass of one or more fungi belonging to the class Zoopagomyceta, Mucoromyceta or Symbiomyceta.

21. Particulate proteinaceous composition according to claim 20, wherein the fungal biomass is biomass of one or more fungi belonging to the phylum Zoopagomycota or Mucoromycota.

22. Particulate proteinaceous composition according to claim 16, wherein the fungal biomass is biomass of a strain of a fungal genus selected from the group consisting of Rasamsonia, Talaromyces, Penicillium, Acremonium, Humicola, Paecilomyces, Chaetomium, Rhizomucor, Rhizopus, Thermomyces, Myceliophthora, Thermoascus, Thielavia, Mucor, Stibella, Melanocarpus, Malbranchea, Dactylomyces, Canariomyces, Scytalidium, Myriococcum, Corynascus, and Coonemeria.

23. Particulate proteinaceous composition according to claim 22, wherein the fungal biomass is biomass of a strain of the genus is Rhizomucor.

24. A process or preparing a particulate proteinaceous composition according to claim 16, said process comprising:
providing fungal biomass;
subjecting the fungal biomass to size reduction treatment to produce a powder or a suspension comprising particles of fungal biomass; wherein the particle size distribution of the particles of fungal biomass meets the following condition:
$$D 10 - prtcl \leq 30 μm ;$$
$$2 μm \leq D 50 - gprtcl \leq 60 μm ;$$
$$D 90 - prtcl \leq 150 μm ;$$
agglomerating the particles of fungal biomass;
wherein the particle size distributions are determined using a Mastersizer 3000 equipped with an Aero S dry powder dispenser; and wherein the particle size distribution of the granules is determined by employing a dispersion pressure of 1 bar and wherein the particle size distribution of the particles of fungal biomass comprised in the granules is determined by employing a dispersion pressure of 4 bar.

25. Process according to claim 24, wherein the fungal biomass is provided in the form of a particulate composition having a D50 of at least 80 μm or in the form of a suspension comprising suspended material having a D50 of at least 80 μm.

26. Process according to claim 24, wherein the size reduction treatment comprises milling or grinding.

27. Process according to claim 24, wherein the size reduction treatment produces a powder comprising the particles of fungal biomass.

28. Process according to claim 24, wherein the particles of fungal biomass are agglomerated by means of fluid bed granulation, compaction, extrusion or spray drying agglomeration.

29. A food product comprising, calculated by weight of dry matter, at least 1 wt. % of the particulate proteinaceous composition according to claim 16.

30. The food product according to claim 29, the food product comprising, calculated by weight of dry matter, 10-95 wt. % of a particulate proteinaceous composition comprising at least 80 wt. % of granules having a protein content of at least 30 wt. % and comprising at least 80 wt. % of particles of fungal biomass, wherein the particle size distribution of the granules meets the following condition:
$$D 50 - grnl \geq 50 μm ;$$
Dx-grnl representing the weight percentage of the granules having a diameter of less than x μm;
wherein the particle size distribution of the particles of fungal biomass comprised in the granules meets the following conditions:
$$D 10 - prtcl \leq 30 μm ;$$
$$2 μm \leq D 50 - gprtcl \leq 60 μm ;$$
$$D 90 - prtcl \leq 150 μm ;$$
Dx-prtcl representing the weight percentage of the particles of fungal biomass comprised in the granules that have a diameter of less than x μm; and
wherein the ratio D50-grnl:D50-prtcl≥2.0;
wherein the particle size distributions are determined using a Mastersizer 3000 equipped with an Aero S dry powder dispenser; and wherein the particle size distribution of the granules is determined by employing a dispersion pressure of 1 bar and wherein the particle size distribution of the particles of fungal biomass comprised in the granules is determined by employing a dispersion pressure of 4 bar.

31. A method of preparing a food product according to claim 30, said method comprising combining a particulate proteinaceous composition comprising at least 80 wt. % of granules having a protein content of at least 30 wt. % and comprising at least 80 wt. % of particles of fungal biomass, wherein the particle size distribution of the granules meets the following condition:
$$D 50 - grnl \geq 50 μm ;$$
Dx-grnl representing the weight percentage of the granules having a diameter of less than x μm;
wherein the particle size distribution of the particles of fungal biomass comprised in the granules meets the following conditions:
$$D 10 - prtcl \leq 30 μm ;$$
$$2 μm \leq D 50 - gprtcl \leq 60 μm ;$$
$$D 90 - prtcl \leq 150 μm ;$$
Dx-prtcl representing the weight percentage of the particles of fungal biomass comprised in the granules that have a diameter of less than x μm; and
wherein the ratio D50-grnl:D50-prtcl≥2.0;
wherein the particle size distributions are determined using a Mastersizer 3000 equipped with an Aero S dry powder dispenser; and wherein the particle size distribution of the granules is determined by employing a dispersion pressure of 1 bar and wherein the particle size distribution of the particles of fungal biomass,
with one or more other edible ingredients.
